(19) United States
(12) Patent Application Publication
(10) Pub. No.: US 2023/0049291 A1
(43) Pub. Date: Feb. 16, 2023

(54) MEDICINE FOR TREATING INFLUENZA OR INFECTIOUS DISEASES

(71) Applicant: Mingchun XIAO, Guangdong (CN)

(72) Inventor: Mingchun XIAO, Guangdong (CN)

(21) Appl. No.: 17/783,661

(22) PCT Filed: Feb. 22, 2020
(86) PCT No.: PCT/CN2020/076289

§ 371 Date: Jun. 9, 2022

(51) Int. Cl. A61K 36/8988; A61K 36/8888; A61K 36/752; A61K 36/74; A61K 36/906; A61K 36/575; A61P 31/10

(52) U.S. Cl. CPC A61K 36/8988; A61K 36/8888; A61K 36/752; A61K 36/74; A61K 36/906; A61K 36/575; A61P 31/10

(57) ABSTRACT

The present invention relates to the field of medical care, and it relates to the application of alkaloids and volatile oils contained in plants such as Gastrodia elata, Pinellia ternata, Orange Peel and Uncaria rhynchophylla in the preparation of medicines or health products for treating influenza or infectious diseases caused by viruses or fungi; especially, it relates to new use of the alkaloids and volatile oils contained in raw materials such as Gastrodia elata, Pinellia ternata, Orange Peel, Unearia and other plants in the preparation of drugs or health products for treating influenza or infectious diseases caused by influenza viruses (including HN avian influenza), coronavirus, Ebola virus, AIDS, Aspergillus, Cryptococcus, Mucor, Histoplasma capsulatum, etc.

### TECHNICAL FIELD

The invention belongs to the field of medical care, and relates to the application of alkaloids and volatile oils contained in plants such as Gastrodia elata, Pinellia ternata, Orange Peel and Uncaria rhynchophylla in the preparation of medicines or health products for treating influenza or infectious diseases caused by viruses or fungi; especially, it relates to new use of the alkaloids and volatile oils contained in raw materials such as Gastrodia elata, Pinellia temata, Orange Peel, Uncaria and other plants in the preparation of drugs or health products for treating influenza or infectious diseases caused by influenza viruses (including HN avian influenza), coronavirus, Ebola virus, HIV, Aspergillus, Cryptococcus, Mucor, Histoplasma capsulatum, etc,

### BACKGROUND OF THE INVENTION

Influenza, also flu for short, is an acute respiratory infectious disease caused by an influenza virus. Influenza viruses belong to Orthomyxoviridae in the virus classification, and are characterized in that their onset is acute, the spread is rapid, and they often cause epidemics or even global pandemics. Clinically, there are fever, fatigue, dry throat pain, joint pain, headache and other toxic symptoms accompanied by respiratory tract infections of varying degrees.

An influenza virus is an enveloped RNA virus. Most of the virus particles are spherical or nearly spherical, and a few are slender or filainentous (such as the influenza virus at the time of initial isolation), It consists of a long nucleic acid chain and a protein shell, The outer layer of proteins is known as a viral coat, and the protein shell contains the genetic material of the virus, which can be DNA or can be RNA. The virus itself can't complete the metabolism, also can't complete the reproduction, and needs to parasitize in other cells to complete.

The influenza virus nucleoprotein antigen is divided into three types: A, B and C. According to the antigenic difference of hemagglutinin (H) and neuraminidase (N) as surface antigens, the homotype virus can be divided into several subtypes. For example, influenza caused by the mutation of HN avian influenza.

Coronaviruses relate to a large class of viruses with a spherical shape. The patients have different clinical symptoms from common cold to a severe pulmonary infection. For example, severe acute respiratory syndrome (SARS is also known as “atypical pneumonia”) in 2003, as well as novel coronavirus 2019-COVID discovered in Wuhan in 2019 and Middle East respiratory syndrome (DEERS) belong to coronaviruses. The main clinical symptoms of SARS virus are fever, physical discomfort, chills, headache, dry throat pain, conjunctivitis, dry cough or dyspnea. The disease progresses rapidly and is prone to systemic infection symptoms and death.

According to experts, the virus of the pneumonia caused by novel coronavirus in 2019 and the virus of the atypical pneumonia in 2003 belong to coronaviruses, wherein the similarity of the gene sequences of these two is 85%. According to Tong Xiaolin, an academician of the Chinese Academy of Sciences, the pneumonia caused by novel coronavirus is a “cold-damp (plague) epidemic”, and is caused by cold-damp seasonal infectious disease. It was found. that whether it was critically ill patients in ICU or patients with mild symptoms in general ward, whether the tongue coating was yellow or white, the overall appearance was thick, greasy and rotten, and the image of dampness was very heavy. He said that pneumonia caused by novel coronavirus is a yin disease by its nature. Therefore, in the treatment of the disease, it must be aimed at cold and dampness, wherein to treat cold pathogen, it is necessary to disperse warm and to relieve superficies, so methods using acrid-warm herbs relieving superficies are used; to treat damp pathogen, aromatics are necessary to avoid turbidity. According to the description of clinical symptoms in the “Diagnosis and Treatment Protocol for Novel Coronavirus Pneumonia” (Trial Version 5), most of the patients mainly have fatigue with gastrointestinal discomfort or fatigue with fever, muscle aches, dry cough, and throat discomfort. After a week, the patients' condition progressively worsens, and pneumonia, even severe pneumonia, and coma appear.

Since the discovery of viruses in 1898, more than 4000 kinds of viruses have been discovered and studied by human beings, but the current answer as to how many types of viruses are in nature is “I don't know”. In the world, Ebola virus, MERS virus, Nipah virus, etc. are infectious diseases caused by new pathogens. The shape of Ebola virus is linear (or of worm type), and its main clinical manifestations are neuropsychiatric symptoms of brain parenchymal damage such as fever, hemorrhage and cerebral infarction. The virus attacks blood vessel wall cells, thereby causing extensive and massive hemorrhage in the viscera, five sense organs, skin mucous membrane and brain, and then due to the continuous massive replication of the virus in blood cells, it causes blood cells to die and coagulate into clots which can block blood vessels widely, thereby causing severe hypoxic-ischemic necrosis and liquefaction of main organs and tissues including the brain. Patients often die rapidly due to shock, poisoning and failure of various organs.

The current mainstream view is that by breaking the mechanism of virus invasion into host cells and sequencing the genes, it is possible to guide the design of drugs, antibodies or vaccines for prevention and control. At present, antiviral medicines mainly include virustat, ribavirin, Tamiflu, etc., but they are prone to drug resistance, and there are many kinds of known viruses, and there are also gene mutations, so the existing antiviral medicines do not have broad-spectrum antiviral effect, and it is difficult for their curative effect to meet the clinical needs.

The main difference between bacteria, fungi and viruses in modern medicine lies in their structures.

The virus is a non-cellular life form, composed of a long nucleic acid chain and protein shell. It has no complete cell structure and contains a single nucleic acid type (DNA or RNA). It is a non-cellular organism that must parasitize within cells and multiply by replication. Most viruses are afraid of heat, but not of cold. They can be inactivated at 56 degrees for 30 minutes or 100 degrees for a few seconds, but they can be kept in a low-temperature refrigerator at −70 degrees or in a state of hypoxia for several months to several years.

Bacteria are unicellular organisms belonging to prokaryotype cells. They are small in size and simple in structure. They have not formed nucleus, nucleolus and nuclear membrane, and have no other organelles than nucleoprotein.

Fungi are enkaryotic microorganisms with relatively complete cell structure, cell walls and typical nuclei; most of them are multicellular, while few of them are unicellular. The unicellular fungi are round or oval, and common unicellular pathogenic fungi mainly include Cryptococcus neoformans and Candida albicans and multiply by germination. The multicellular fungi have mycelia and spores, wherein the hyphae are interwoven into clusters called filamentous fungi and mould. The common pathogenic fungi are mainly Aspergillus and Mucor. In addition, there are biphasic fungi which can survive in the form of hyphae and yeast under certain conditions and are important pathogenic fungi in human beings, and the most common of them are Histoplasma capsulatum. The physiological characteristics of fungi is that they are also afraid of heat, but not of cold, wherein the hyphae and spores are killed at 60 degrees in 1 hour. Fungi have strong resistance to dryness, sunlight, cold and oxygen lack, In addition, fungi are widely distributed in nature. At present, there are more than about 70000 species of fungi described in the world, and there are more than 300 fungi identified as pathogenic bacteria today.

Studies have shown that the nucleus of yeast is an organelle with double membrane structure. The chromosome in the nucleus is composed of DNA and histones. There is one or several regions in the nucleus with high RNA content. This region is the nucleolus, which is the site of ribosome synthesis. The centrosome is located outside the nuclear membrane, is a filamentous structure composed of protein subunits, and plays a role in cell reproduction and division. DNA sequencing of gene sequencing is the main development direction at present.

Medical researches in recent years have also proved that the main route of aspergillosis infection is to inhale spores in the air. Aspergillus spores enter the sinuses and lungs during inhalation, germinate to produce hyphae and enter cytoplasmic changes, and Aspergillus likes to invade blood vessels. Inhalation of large amount of Aspergillus spores through the respiratory tract can cause acute pneumonia. The patient's condition is severe. The failure to treat it in time can often lead to death. Its clinical symptoms are chills, fever, fatigue, irritating cough, cough with yellow sputum, sometimes with blood, rapid symptoms of pneumonia, and dyspnea.

In recent years, medical researches have also proved that the main transmission routes of fungi such as Cryptococcosus and Mucor are the respiratory tract and skin, which can cause systemic cryptococcosis, rhinocerebral mucormycosis, pulmonary mucormycosis, disseminated mucormycosis, etc. According to the record in “Medical Mycology” edited by Wen Hai (People's Medical Publishing House, October 2012, 1^{st} Edition), cryptococcosis is deep mycosis caused by some species or varieties of Cryptococcus, which mainly invades the central nervous system, lungs and bones. Its first clinical symptoms are symptoms of upper respiratory tract infection such as chills, fever, headache, dizziness, sore throat, conjunctivitis, nasal congestion, nausea, vomiting, general discomfort and other symptoms, wherein the general treatment is not effective, the symptoms gradually worsen, the mortality rate is high, the prognosis is serious. At present, more than 70 species of Cryptococcus are known, and new varieties of Cryptococcus neoformans are widely distributed all over the world. Almost all cryptococcal infections in AIDS patients are caused by this variety, and the early symptoms of AIDS are similar to those of colds.

Mucor and Entomophthora are collectively referred to as zygomycosis. Because Mucor is the majority, some scholars use Mucor as a substitute for zygomycosis. Mucor and Entomophthora can be spread through air, dust and food. They tend to invade artery blood vessels and causes thrombosis, tissue ischemia, infarction and necrosis. The spores of Mucor are spherical in shape, and the genera of Entomophthora include C. coronatus and C. incongruus.

Histoplasma capsulatum forms hypha phase when cultured at room temperature and in soil, and transforms into yeast phase when cultured at 37 degrees or infecting a host, It mainly invades mononuclear phagocytic system or lung. The disease is mainly prevalent in America and Africa. The respiratory tract is the main route of infection, which is caused by inhalation of fungal spores from birds or contaminated soil. The clinical symptoms are those of influenza, such as fever, chills, headache, dry cough, chest pain, etc. Several outbreaks of the disease have been reported in the United States.

The patent “Cracking the Pathogenic Factors of Cancer and Traditional Chinese Medicine Treatment Method” to the application filed earlier by the applicant (the application No. 201310274489.7), as well as the international patent “A Pharmaceutical Composition for the Treatment of Cancer and Preparation Method thereof” (the application No. PCT/CN2013/080936, the application No. 201380075486.9 for entry into national phase in China, the patent No. U.S. Pat. No. 9,498,508B2 for entry into the United States) refer to the “damp pathogen” mentioned in traditional Chinese medicine, which refers to the pathogenic bacteria, that can survive in blood, mucus and other liquids, mainly refers to mould, viruses, bacilli and other pathogenic bacteria that can survive in the state of hypoxia; the “cold pathogen” refers to mould; and the “wind pathogen” refers to the pathogenic bacteria, that can be transmitted through air. The international patent “A Pharmaceutical Composition for Preventing and Treating Cancer of Reproductive System and Preparation Method thereof” (the application No. PCT/CN2013/08481.3, the application No. 201380053736.9 for entry into national phase in China, the patent No. U.S. Pat. No. 9,744,206B2 for entry into the United States) also refers to a method for treating common cold due to wind-cold by using the properties of the “Banxia Baizhu Tianma Decoction” against fungi, viruses and bacilli. The divisional application “Medicine for Treating Cold Caused by Fungi” (application No. 201910444433.9) refers to the composition with Gastrodia elata and Pinellia ternata as the sovereign drug that can treat common cold due to wind-cold. There is also a divisional application “Application of the Dang Gui Zhi Tong Decoction in the Preparation of Antimould Drugs or Health Products” (application No. 201811463563.9) which refers to a new use of the “Dang Gui Zhi Tong Decoction” in the preparation of drugs or health products for treating influenza caused by infection of mould. There are also international patents “Medicine for Treating Strokes Caused by Deep Fungal Infections” (application No. PCT/CN2019/099352), “Medicine for Treating Pulmonary Mycosis” (application No. PCT/CN2019/102246) and “Medicine for Treating Digestive System Mycosis” (application No. PCT/CN2019/125796) that refer to the application of alkaloids and/or volatile oils contained in Gastrodia elata and Pinellia ternata in the preparation for treating fungal diseases. Based thereon, this application is the continuation application.

The “Banxia Baizhu Tiamna Decoction”, “Er Chen Decoction” and “Yu Hu Pill” all have expectorant effect. The raw material of the “Banxia Baizhu Tianma Decoction” consists of 4.5 g of Pinellia ternata, 9 g Atractylodes macrocephala, 3 g of Gastrodia elata, 3 g of Poria cocos, 3 g of Tangerine Peel, 1.5 g of Licorice, 1 piece of Ginger and 2 pieces of Jujube. The raw material of the “Er Chen Decoction” consists of 15 g of Pinellia ternata, 15 g of Tangerine Peel, 9 g of Poria cocos and 4.5 g of Liquorice, wherein 7 pieces of Ginger and one Smoked Plum are further added. The raw material of the “Yu Hu Pill” consists of one Jiang of Pinellia ternata, 5 qians of Gastrodia elata, one liang of Sheng Nanxing, plus Ginger for delivery. The shortcoming of traditional classical prescriptions is that the effective chemical components are not clear. The preparation methods of modern Chinese patent medicines are usually the methods for preparing medicines by mixing all the raw materials and boiling them together, and then extracting them. The main shortcoming of these methods is that it is possible to know only the main ingredients, rather than all the ingredients.

Pinellia ternata is a drug for warming and resolving cold phlegm, and it belongs to the spleen, stomach and lung meridians. It is mainly used to treat the syndromes of damp phlegm and cold phlegm, as well as vomiting. According to modem studies, Pinellia temata contains volatile oils and alkaloids, wherein its alkaloids have anti-tumor and cough stopping effects. There are many reports on the determination of total alkaloid content in Pinellia ternata in China and abroad. Pinellia ternata alcohol extract has broad-spectrum. antibacterial activity and has a certain effect regarding inhibitory action on fungi.

Gastrodia elata is a drug for relieving wind and spasm, and it belongs to the liver meridian. It is a medicine for treating headache, numbness of the limbs, rheumatoid. arthritis, blood pressure drop, etc. According to the research by Guanping et al. [Journal of Southwest Normal University Natural Science Edition), 2008], it is found that the volatile oil of Gastrodia elata f. glauca has a significant inhibitory action on Aspergillus oryzae and Aspergillus flavus in agricultural products, and the volatile oil of Gastrodia elata f. glauca has a significant antibechic and expectorant effects.

Orange Peel is a Qi regulating drug, and it belongs to the spleen and lung meridians. According to modern researches, the decoction of fresh Orange Peel has the effect of trachea dilation, the volatile oil has a stimulating effect on phlegm removal, and the total water-soluble alkaloids of Dried Orange Peel have the effect of raising blood pressure. The volatile oil content of Dried Orange Peel is 1.9%-3.5%, and the alkaloid content of Dried Orange Peel is 0.240% to 0.636%.

Uncaria rhynchophylla and Gastrodia elata are both drugs for calming wind and relieving spasm, and they belong to the liver and pericardium meridians. They are used. to treat headache and dizziness. They contain a variety of alkaloids and have antihypertensive, antiepileptic and antiarrhythmic effects. However, there are no reports on the antiviral and antifungal effects of the alkaloids of Uncaria rhynchophylla.

Many scholars in China and abroad have found that alkaloids have many pharmacological activities, including anti-tumor, anti-virus, anti-inflammatory, antibacterial, analgesic, liver protection, cardiovascular and many other aspects. However, at present, the pharmacological. mechanisms of anti-tumor, anti-virus, anti-inflammatory, antibacterial, analgesic, liver protection, cardiovascular effects of alkaloids have not been fully studied. Furthermore, because the pharmacological mechanism of alkaloids has not been studied clearly in the existing technology, and the synergistic effect between various alkaloids is also unknown, the existing pharmaceutical chemicals are produced from single alkaloids contained in single plants. The shortcoming of single alkaloid drugs is that the dose of a single alkaloid is too large, and toxic side reactions are readily observed.

Modern pharmacological experiments on volatile oils show that: volatile oils have anti-inflammatory, anti-allergic, antimicrobial, anti-imitation, anti-cancer effects, anthelmintic effects, enzyme inhibition effects, effects on the central nervous system and effects on the respiratory system. However, the understanding and research on the activity mechanism or pharmacological mechanism of volatile oils are still very limited. Modern studies have also shown that some volatile oils have inhibitory action on fungi.

### SUMMARY OF THE INVENTION

The inventor's research on the existing technology of coronavirus and Cryptococcus show that coronavirus and Cryptococcus neoformans have many similarities, their clinical symptoms are highly identical, the shape is spherical, the virus structure contains a single nucleic acid (DNA or RNA) type, wherein Cryptococcus is also a unicellular structure containing DNA and RNA. Their physiological characteristics are the same. They are afraid of heat, but not of cold. They can be inhibited or killed at 50-60 degrees, and can survive in the conditions of low temperature and oxygen lack. C. coronatus is also a coronal form. The influenza virus (HN avian influenza) is also highly similar to Histoplasma capsulatum. Ebola virus is also highly similar to that of Entomophthora, and Cryptococcus is also found in AIDS patients. Therefore, the invention continues to optimize pharmaceutical formulation based on the “Banxia Baizhu Tianma Decoction”, “Er Chen Decoction” and “Yu Hu Pill”.

The purpose of the invention is to provide a medicine for treating influenza or infectious diseases caused by viruses or fungi, in particular to provide new use of alkaloids and volatile oils contained in plants such as Gastrodia elata, Pinellia ternata, Orange Peel and Uncaria rhynchophylla in the preparation of drugs or health products for treating influenza or infectious diseases caused by viruses or fungi.

Furthermore, the medicine for treating influenza or infectious diseases provided by the invention is characterized in that the active pharmaceutical ingredients of the medicine include total alkaloids and/or total volatile oils extracted from Gastrodia elata, total alkaloids and/or total volatile oils extracted from Pinellia ternata, total alkaloids and/or total volatile oils extracted from Orange Peel, and total alkaloids and/or total volatile oils extracted from Uncaria rhynchophylla.

Furthermore, the medicine provided by the invention is characterized in that the weight parts of the active pharmaceutical ingredients of the medicine are: 0.01-50 weight parts of the total alkaloids and/or the total volatile oils extracted from Gastrodia elata, 0.01-50 weight parts of the total alkaloids and/or the total volatile oils extracted from Pinellia temata, 0.01-50 weight parts of the total alkaloids and/or the total volatile oils extracted from Orange Peel, 0.01-50 weight parts of the total alkaloids and/or the total volatile oils extracted from Uncaria rhynchophylla.

Furthermore, the dosage of each raw material of the medicine can be determined with reference to the dosage of traditional Chinese medicine decoction, and the weight of the fresh products of the raw materials of the medicine is preferably 20-250 g of Gastrodia elata, 20-250 g of Pinellia ternata, 20-250 g of Orange Peel and 20-250 g of Uncaria rhynchophylla. Generally, 350 g-450 g of fresh products can be made into about 100 g of dry products.

Furthermore, the dosage of each raw material of the medicine can be determined with reference to the dosage of traditional Chinese medicine decoction, and the weight of the dry products of the raw materials of the medicine is preferably 5-50 g of Gastrodia elata, 5-50 g of Pinellia ternata, 5-50 g of Orange Peel and 5-50 g of Uncaria rhynchophylla.

Furthermore, according to clinical symptoms, the medicine for treating influenza or infectious diseases provided by the invention can be selectively added with active pharmaceutical ingredients, which is characterized in that when the clinical symptoms appear to be upper respiratory tract infection symptoms such as chills, fever or no fever, headache, dizziness, sore throat, conjunctivitis and nasal obstruction, the active pharmaceutical ingredients of the medicine can be selectively added with: total alkaloids and/or total volatile oils extracted from Ginger, total alkaloids and/or total volatile oils extracted from Notopterygium incisum, total alkaloids and/or total volatile oils extracted from Saposhnikovia divaricata, total alkaloids and/or total volatile oils extracted from Ligusticum levisticum, total alkaloids and/or total volatile oils extracted from Flos Magnoliae, total alkaloids and/or total volatile oils extracted from Mentha haplocalyx, total alkaloids and/or total volatile oils extracted from Mulberry Leaves, total alkaloids and/or total volatile oils extracted from Chaste Tree Fruits.

Furthermore, when the clinical symptoms appear to be fatigue, vomiting, diarrhea, constipation and other digestive system symptoms, the active pharmaceutical ingredients of the medicine can be selectively added with: total alkaloids and/or total volatile oils extracted from Agastache rugosa, total alkaloids and/or total volatile oils extracted from Atractylodes lancea, total alkaloids and/or total volatile oils extracted from Magnolia officinalis.

Furthermore, when the clinical symptoms appear to be pneumonia, dyspnea and asthma, the active pharmaceutical ingredients of the medicine can be selectively added with: total alkaloids and/or total volatile oils extracted from Rhizoma arisaematis, total alkaloids and/or total volatile oils extracted from Sinapis alba, total alkaloids and/or total volatile oils extracted from Fritillaria cirrhosa, total alkaloids and/or total volatile oils extracted from Trichosanthes kirilowii, total alkaloids and/or total volatile oils extracted from Radix Stemona, and total alkaloids and/or total volatile oils extracted from Semen Lepidii.

Furthermore, when the clinical symptoms appear to be general symptoms, the active pharmaceutical ingredients of the medicine can be selectively added with: total alkaloids and/or total volatile oils extracted from Radix angelicae pubescentis, total alkaloids and/or total volatile oils extracted from Ligusticum wallichii, total alkaloids and/or total volatile oils extracted from Achyranthes bidentata, total alkaloids and/or total volatile oils extracted from Astragalus membranaceus, total alkaloids and/or total volatile oils extracted from Angelica sinensis, total alkaloids and/or total volatile oils extracted from Aconitum carmichaeli, total alkaloids and/or total volatile oils extracted from Herba artemisiae scopariae, total alkaloids and/or total volatile oils extracted from Garlic, total alkaloids and/or total volatile oils extracted from Cirsium setosum, and total alkaloids and/or total volatile oils extracted from Cirsium japonicum.

Furthermore, method 1 for preparing the medicine of the invention is as follows: taking the total alkaloids of Gastrodia elata, the total alkaloids of Pinellia ternata, the total alkaloids of Orange Peel and the total alkaloids of Uncaria rhynchophylla and mixing them in a certain proportion; adding various conventional excipients required to prepare different dosage forms thereto; and preparing pharmaceutically acceptable dosage forms.

Method 2 for preparing the medicine is as follows: taking the total volatile oils of Gastrodia elata, the total volatile oils of Pineilia ternata, the total volatile oils of Orange Peel and the total volatile oils of Uncaria rhynchophylla and mixing them in a certain proportion; adding various conventional excipients required to prepare different dosage forms thereto; and preparing pharmaceutically acceptable dosage forms.

Method 3 for preparing the medicine is as follows: taking the total alkaloids and volatile oils of Gastrodia elata, the total alkaloids and volatile oils of Pineilia ternata, the total alkaloids and volatile oils of Orange Peel, the total alkaloids and volatile oils of Uncaria rhynchophylla; mixing the volatile oils in a proportion, then mixing them with the dry alkaloid substances and an appropriate amount of excipients in a certain proportion after inclusion with beta-cyclodextrin, and preparing pharmaceutically acceptable dosage forms using conventional methods for preparing medicines.

Furthermore, in the preparation methods, the methods for extracting the alkaloids can be determined after extraction with modern commonly used organic solvents, and the alkaloids are extracted respectively from raw materials such as Gastrodia elata, Pinellia ternata, Orange Peel and Uncaria rhynchophylla using appropriate solvents. Supercritical fluid extraction is the preferred choice, followed by membrane separation, ultrasonic extraction or Soxhlet extraction, etc.

Furthermore, in the preparation methods, the methods for extracting the volatile oils can adopt modern commonly used extraction methods to extract the volatile oils from raw materials such as Gastrodia elata, Pinellia ternata, Orange Peel and Uncaria rhynchophylla respectively. Supercritical fluid extraction or steam distillation are the first choice, followed by organic solvent extraction: continuous reflux extraction or cold leaching using petroleum ether, diethyl ether and other organic solvents; a cold pressing method or steam distillation are preferred for extraction from fresh Orange Peel.

Furthermore, the medicine is characterized in that the specific preparation method based on the active pharmaceutical ingredients added or subtracted according to the clinical symptoms includes: extracting the alkaloids from each of the raw materials, mixing the alkaloids in a proportion; adding various conventional excipients required to prepare different dosage forms thereto, and preparing pharmaceutically acceptable dosage forms such as tablets, powder injections, capsules, granules using conventional methods for preparing medicines.

Furthermore, the medicine is characterized in that the specific preparation method based on the active pharmaceutical ingredients added or subtracted according to the clinical symptoms includes: extracting the volatile oils from each of the raw materials, mixing the volatile oils in a proportion; adding various conventional excipients required to prepare different dosage fours thereto, and preparing pharmaceutically acceptable dosage forms such as tablets, soft capsules, topical liniments, granules using conventional methods for preparing medicines.

Furthermore, the medicine is characterized in that the specific preparation method based on the active pharmaceutical ingredients added or subtracted according to the clinical symptoms includes: extracting the alkaloids and volatile oils from each of the raw materials, mixing the volatile oils in a proportion; then mixing them with the dry alkaloid substances and an appropriate amount of excipients after inclusion with beta-cyclodextrin; and preparing pharmaceutically acceptable dosage forms such as tablets, granules using conventional methods for preparing medicines.

Further, the medicine for treating influenza or infectious diseases is characterized in that the medicine has broad-spectrum antiviral or broad-spectrum antifungal actions on but not limited to, for example, influenza viruses (including ITN avian influenza virus), coronaviruses (including novel coronavirus 2019-COVID, MERS virus, SARS coronavirus), Ebola virus, AIDS, Aspergillus, Cryptococcus, Mucor, Entomophthora, Histoplasma capsulatum.

The pharmacology of the raw materials of the medicine of the invention is as follows:

Uncaria rhynchophylla and Gastrodia elata are drugs for calming wind and relieving spasm, and they belong to the liver meridian. The wind refers to the “wind pathogen”. Ancient Chinese doctors called pathogenic factors “the six external factors which cause diseases”: wind, cold, heat, damp, dryness, fire. The ancients did not have modern testing instruments, and the judgement about things depended on the senses. Therefore, the inventor thinks that the classification of pathogenic factors by the ancients was also based on the principle of sensibility. From the symptoms described in traditional Chinese medicine, it can be concluded that the “wind pathogen” refers to the pathogenic bacteria that can be transmitted by air; the “cold pathogen” refers to the pathogenic bacteria that cause shivers and cold in the body; the “damp pathogen” refers to the pathogenic bacteria that can survive in blood, mucus and other liquids; the “heat pathogen” refers to the pathogenic bacteria that can cause summer heat stroke symptoms; the “dryness pathogen” refers to the pathogenic bacteria that easily cause dry mouth, lung dryness, etc.; and the “fire pathogen” refers to the pathogenic bacteria that can cause the body to have a high fever. From modern medical researches, it is known that viruses can cause cold by droplet infection of the upper respiratory tract, and fungi (such as Cryptococcus, Aspergillus, Mucor, Histoplasma capsulatum) can also cause droplet infection of the upper respiratory tract through spores or bacteria contained in the air. Therefore, Gastrodia elata and Uncaria have antiviral or antifungal effects, thereby preventing influenza or infectious diseases.

Modern researches showed that volatile oil of Gastrodia elata f. glauca has significant antibechic and good expectorant effects. Because viruses and fungi can infect the whole body through disseminated blood, coronavirus, Cryptococcus, Histoplasma capsulatum and so on cause infection mainly through the upper respiratory tract. Their characteristic is that they are prone to invade blood vessels, and as there are abundant blood vessels in the brain, the brain is also a place where viruses and fungi are easy to stay, which will cause symptoms such as headache and dizziness. Because of the blood-brain barrier, many medicines can't enter the brain, while Gastrodia elata and Uncaria rhynchophylla are medicines that can pass through the blood-brain barrier.

The medicinal properties of Gastrodia elata are sweet and flat, and the medicinal properties of Uncaria rhynchophylla are sweet and cool. They can be used in case of fever or no fever, and have broad-spectrum antiviral and antifungal actions. Gastrodia elata and Uncaria rhynchophylla belong to the liver meridian. The inventor thinks that the liver meridian comes out of the group of the portal veins of the liver. Gastrodia elata and Uncaria rhynchophylla can also treat viruses and fungi in the liver. The combined effect of the two can enhance the curative effect. Alkaloids and volatile oils are the main antiviral and antifungal active ingredients of Gastrodia elata and Uncaria rhynchophylla.

Pinellia ternata is a drug for warming and resolving cold phlegm. The medicinal properties is pungent and warm. It is mainly used for treating damp phlegm and cold phlegm syndromes. This shows that Pinellia, ternata can treat the “damp pathogen” and the “cold pathogen”. The inventor thinks that the “damp pathogen” is a variety of bacteria that live in blood (veins) or mucus and are tolerant to oxygen lack (such as fungi, viruses, anaerobic bacteria, bacilli, etc.), while the “cold pathogen” is fungi (such as Candida albicans). Pinellia ternata can remove dampness and expel cold. Pinellia temata can act on viral and fungal infections of lower respiratory tract, as well as on viral and fungal infections of digestive system. It has the effect of relieving cough, resolving phlegm and stopping vomiting. When the patient has a fever, it can be combined with drugs for clearing away heat phlegm such as Fritillaria cirrhosa and Trichosanthes kirilowii. When dyspnea occurs, the combination of Rhizoma arisaematis and Sinapis alba can enhance the antiviral and antifungal drug effects. Therefore, Pinellia temata has broad-spectrum antiviral or antifungal actions. Traditional Chinese medicine adheres to the principle of “removing dampness by means of aromatics, removing dampness by means of pungent, removing coldness by means of warm”. The aroma and pungent taste of plants come from their volatile oils. Mould likes acidic and alkalescent conditions. The alkaloids have antiviral effect, so the antiviral or antifungal active ingredients of Pinellia ternata are alkaloids and volatile oils. Orange Peel is a Qi regulating drug, and its medicinal properties are pungent, bitter and warm. It has the effect of regulating Qi for relieving pain. Regulating Qi promotes the exchange of oxygen and carbon dioxide. Because a viral or fungal infection of the respiratory tract will cause massive inflammatory edema, resulting in difficult exchange of oxygen and carbon dioxide. A viral or fungal infection of the gastrointestinal tract easily leads to carbon dioxide retention in the gastrointestinal tract and thus to gas and a distending pain in the gastrointestinal tract. Orange Peel has the function of promoting peristalsis, so that the carbon dioxide is expelled. Alkaloids and volatile oils are the main antiviral and antifungal active ingredients of Orange Peel.

Ginger, Notopterygium incisum, Saposhnikovia divaricata, Ligusticum levisticum and Flos Magnoliae are wind-cold dispersing drugs with pungent and warm medicinal properties. Traditional Chinese medicine divides cold into common cold due to wind-cold and common cold due to wind-heat. When influenza patients have chills, low fever, cold, headache, nasal congestion, anhidrosis, aches in the limbs and other clinical symptoms of common cold due to wind-cold, Ginger, Notopterygium incisum, Saposhnikovia divaricata, Ligusticum. levisticum and Flos Magnoliae can be selectively added to help Gastrodia elata and Uncaria rhynchophylla provide the antiviral or antifungal drug effects. The main active ingredients of Ginger, Notopterygium incisum, Saposhnikovia divaricata, Ligusticum levisticum and Flos Magnoliae as wind-cold dispersing drugs are alkaloids and volatile oil.

Mentha haplocalyx, Mulberry Leaves and Chaste Tree Fruits are wind-heat dispersing drugs with pungent, cool or cold medicinal properties. When influenza patients have high fever, headache, dizziness, sore throat, yellow phlegm, dry mouth and other symptoms of common cold due to wind-heat, Mentha haplocalyx, Mulberry Leaves and Chaste Tree Fruits can be selectively added to help Gastrodia elata and Uncaria rhynchophylla provide the antiviral and antifungal drug effects. Alkaloids and volatile oils are the main antiviral and antifungal active ingredients of Mentha haplocalyx, Mulberry Leaves, Chaste Tree Fruits and other wind-heat dispersing drugs.

Agastache rugosa, Atractylodes lancea and Magnolia officinalis are dampness-resolving drugs. Their medicinal properties are pungent and warm. They mainly target viral and fungal infections in the digestive system. After viruses or fungi invade the human body through the nasal cavity, the upper respiratory tract symptoms are the first to appear, then the organs of the digestive system such as the liver are infected through the blood circulation, the digestive tract symptoms appear, and then the pneumonia symptoms appear in the lungs. If viruses or fungi invade the human body through the mouth cavity, the digestive tract symptoms will be the first to appear, and then the viruses or fungi easily enter the brain capillary blood vessels, so that the headache symptoms appear, or enter the lungs, and the pneumonia symptoms appear. Therefore, when fatigue, vomiting, diarrhea, constipation, hypofunction of liver and other symptoms appear, Agastache rugosa, Atractylodes lancea and Magnolia officinalis can be selectively used to help Pinellia ternata enhance the antiviral or antifungal actions. The main active ingredients of Agastache rugosa, Atractylodes lancea and Magnolia officinalis against various viruses or fungi are alkaloids and volatile oils.

Rhizoma arisaematis, Sinapis alba and Pinellia ternata are drugs for warming and resolving cold phlegm. Their medicinal properties are pungent and warm. When the tongue coating is white, thick and greasy, the sputum is white and sticky, and the pneumonia symptoms are serious, Rhizoma arisaematis and Sinapis alba can be added to enhance the expectorant effects of Pinellia ternata and Gastrodia elata. Rhizoma arisaematis is also a medicine that can pass through the brain barrier. Alkaloids and volatile oils are the main antiviral and antifungal active ingredients of Rhizoma arisaematis and Sinapis alba.

Fritillaria cirrhosa and Trichosanthes kirilowii are drugs for clearing away heat phlegm. The medicinal properties are bitter and cold. When the tongue coating is yellow, thick and greasy, and the sputum is yellow and purulent, Fritillaria cirrhosa and Trichosanthes kirilowii can be added to enhance the expectorant effect of Pinellia ternata. Alkaloids and volatile oils are the main antiviral and antifungal active ingredients of Fritillaria cirrhosa and Trichosanthes kirilowii.

Radix Stemona and Semen Lepidii are drugs for treating cough and asthma. The medicinal properties of Radix Stemma are sweet, bitter and slightly warm. It belongs to the lung meridian and contains a variety of alkaloids. Radix Stemma has inhibitory action on influenza and skin fungi. The medicinal properties of Semen Lepidii are bitter, pungent and very cold, and it belongs to the lung meridian and the urinary bladder meridian. When there is chest tightness and asthma, Radix Stemona and Semen Lepidii can be added to enhance the expectorant effect of Pineilia ternata. Alkaloids and volatile oils are the main antiviral and antifungal active ingredients of Radix Stemma and Semen Lepidii.

Radix angelicae pubescentis is a drug for dispelling wind, dampness and cold. That is, Radix angelicae pubescentis can treat the “wind pathogen, damp pathogen and cold pathogen”, so Radix angelicae pubescentis has broad-spectrum antiviral and antifungal actions. When limb pain is serious, especially in case of lower limb joint arthropathy, adding Radix angelicae pubescentis to Gastrodia elata, Uncaria rhynchophylla and Notopterygium incisum enhances their treatment of viral and fungal infections of bones. For example, Cryptococcus easily damages bones. Alkaloids and volatile oils are the main antiviral and antifungal active ingredients of Radix angelicae pubescentis.

Ligusticum wallichii and Achyranthes bidentata are blood-activating drugs. The medicinal properties of Ligusticum wallichii is pungent and warm, and it belongs to the liver, gallbladder and pericardium meridians. The medicinal properties of Achyranthes bidentata are flat, and it belongs to the liver and kidney meridians. When influenza patients develop general symptoms, Ligusticum wallichii and Achyranthes bidentata are added to help Gastrodia elata and Uncaria rhynchophylla enhance their treatment of viral and fungal infections in the heart, cerebral vessels and limb bones. Alkaloids and volatile oils are the main antiviral and antifungal active ingredients of Ligusticum wallichii and Achyranthes bidentata.

Astragalus membranaceus is a Qi-tonifying drug, and its medicinal properties are sweet and slightly warm. In traditional Chinese medicine, it is believed that “spleen deficiency leads to phlegm-dampness”, in other words, viruses or fungi invade liver blood vessels, causing disseminated blood infection. Fungal mass easily blocks blood. vessels, causing difficulty in the exchange of oxygen and carbon dioxide in the blood. “Qi deficiency” is oxygen lack. A Qi-tonifying drug and Qi regulating drug Orange Peel can promote expulsion of carbon dioxide from the respiratory tract and gastrointestinal tract. Astragalus membranaceus is an important drug for tonifying spleen for nourishing Qi. It is used for severe influenza patients with weak constitution and oxygen lack symptoms. Astragalus membranaceus is added to help Pinellia ternata restore lung function. The main chemical ingredients of Astragalus membranaceus against various fungi or viruses are alkaloids, volatile oils and polysaccharides.

Angelica sinensis is a blood-tonifying drug, its medicinal properties are sweet, pungent and warm, and it belongs to the liver, heart and spleen meridians. It is often combined with Qi-tonifying drug Astragalus membranaceus. The main chemical ingredients of Angelica sinensis against various fungi or viruses are alkaloids, volatile oils and polysaccharides.

Aconitum carmichaeli is a drug for dispelling cold, its medicinal properties are pungent and hot, and it belongs to the heart, kidney and spleen meridians. The toxicity of aconitine obtained by hydrolysis of Aconitum carmichaeli can be greatly reduced. The inventor thinks that Candida albicans is a “cold pathogen”, wherein Candida albicans is a conditional pathogen, which is contained in any human body. When the body has strong immunity, its activity is low, but when there is invasion of pathogenic bacteria such as in vitro viruses or fungi or bacilli or bacteria, it will stimulate its intensive reproduction, thus accelerating the development of the disease and easily causing general infection symptoms. Therefore, when the symptoms of renal hypofunction appear, Aconitum carmichaeli is added to help Pinellia ternata and Gastrodia elata enhance the antiviral and antifungal drug effects. Alkaloids and volatile oils are the main antifungal and antiviral active ingredients of Aconitum carmichaeli.

Herba artemisiae scopariae is a drug for removing dampness and jaundice, as well as a diuretic. Its medicinal properties are bitter, pungent and slightly cold, and it belongs to the spleen, stomach, liver and gallbladder meridians. It is used for the treatment of viral or fungal jaundice, and inflammatory edema all over the body. Herba artemisiae scopariae is added to increase the antiviral and antifungal drug effects of Pinellia ternata and Aconitum carmichaeli. The effective antiviral and antifungal chemical ingredients of Herba artemisiae scopariae are volatile oils and alkaloids.

Garlic: the medicinal properties are pungent and warm. It belongs to the spleen, stomach and lung meridians, and it is a drug for detoxification and killing parasites to relieve itching. According to modern researches, the main component of Garlic is volatile oil, and it has relatively strong broad-spectrum antibacterial action and fungal inhibiting action. Garlic has a good effect of promoting gastrointestinal peristalsis, and can help Orange Peel enhance the Qi regulating drug effect. When the constipation symptoms appear, Garlic is added to help Agastache rugosa, Atractylodes lancea and Magnolia officinalis enhance their antiviral and antifungal effects. The effective antiviral and antifungal ingredients of Garlic are volatile oils and alkaloids.

Cirsium setosum and Cirsium japonicum are hemostatic drugs, and its medicinal properties are sweet, bitter and cool, and it belongs to the heart and liver meridians. They can be selectively added when the clinical symptoms of bleeding appear. The effective antiviral and antifungal ingredients of Cirsium setosum and Cirsium japonicum are volatile oils and alkaloids.

The formula principles of the invention are: according to the invention, the total alkaloids and/or volatile oils extracted from Gastrodia data, the total alkaloids and/or volatile oils extracted from Pinellia temata, the total alkaloids and/or volatile oils extracted from Orange Peel and the total alkaloids and/or volatile oils extracted from Uncaria rhynchophylla are used as basic drugs for treating various viral or fungal infections. The basic drugs of the invention have broad-spectrum antiviral or antifungal actions, and also have the action of resisting systemic infections, and they are suitable for influenza or infectious diseases caused by all viruses or fungi. All the raw materials of the invention have antiviral and antifungal effects, but they also have their own characteristics and have different targeting effects. Therefore, they can be selectively added according to different clinical symptoms.

The inventor thinks the pathogenic bacteria world also has the characteristics similar to those of a kingdom, wherein the host is their territory, fungi are the ruling class, and Candida albicans is the king. The inventor thinks the white blood cells in the blood may be the legion of the Candida albicans. After the invasion of external pathogenic bacteria, the fungi in the body will spontaneously organize forces to resist the invaders, that is, “rejection reaction” or “immune response”, in order to maintain their dominant position. For example, during the epidemic caused by the SARS coronavirus in 2003, many atypical pneumonia survivors suffered serious sequelae, and the examinations showed that there were a lot of fungi in their organisms. For example, the invention of penicillin showed that Penicillium can kill other bacteria. The modem medical community has found that Penicillium also has the characteristics of epidemic disease. Penicillium also mainly invades the respiratory tract, wherein the primary symptoms are mainly in the lungs, and the clinical manifestations are similar to those of pulmonary tuberculosis. Therefore, the process when weak fungi become strong in the host body is the process of the host aging and death. Therefore, whether it is a viral infection or other bacterial infection, antifungal treatment is the key when the disease develops to the middle and late stages.

### Specific Implementation Mode

### Example 1

The medicine for treating influenza caused by an influenza virus (including HN avian influenza) or Histoplasma capsulatum or Aspergillus flavus according to this example. The formulation is optimized according to the clinical manifestations of influenza.

1. Typical influenza, also known as simple flu. The clinical manifestations are: sudden chills, fever, headache, eye pain, muscle soreness, obvious fatigue, dry throat pain, burning sensation under sternum (from trachea), the upper respiratory tract symptoms such as occasional nasal congestion, runny nose, sneezing, dry cough, as well as nausea and diarrhea.

The composition of the active pharmaceutical ingredients (dry product) of this example is as follows: total alkaloids and/or volatile oils extracted from 25 g of Gastrodia elata, total alkaloids and/or volatile oils extracted from 15 g of Pinellia ternata, total alkaloids and/or volatile oils extracted from 10 g of Orange Peel, total alkaloids and/or volatile oils extracted from 25 g of Uncaria rhynchophylla, total alkaloids and/or volatile oils extracted from 10 g of Notopterygium incisura, total alkaloids and/or volatile oils extracted from 15 g of Saposhnikovia divaricata, total alkaloids and/or volatile oils extracted from 15 g of Flos Magnoliae, total alkaloids and/or volatile oils extracted from 20 g of Mentha haplocalyx, total alkaloids and/or volatile oils extracted from 20 g of Mulberry Leaves, total alkaloids and/or volatile oils extracted from 15 g of Ligusticum wallichii, total alkaloids and/or volatile oils extracted from 20 g of Atractylodes lancea.

2. Pneumonic influenza, also known as primary influenza virus pneumonia.

(1) Mild onset such as typical influenza: cough, light grey sticky sputum after 1-2 days, no obvious dyspnea, X-ray examination of the lungs shows inflammatory shadow.

The composition of the active pharmaceutical ingredients (dry product) of this example is as follows: total alkaloids and/or volatile oils extracted from of 25 g of Gastrodia elata, total alkaloids and/or volatile oils extracted from 25 g of Pinellia ternata, total alkaloids and/or volatile oils extracted from 20 g of Orange Peel, total alkaloids and/or volatile oils extracted from 20 g of Uncaria rhynchophylla, total alkaloids and/or volatile oils extracted from 20 g of Sinapis alba, total alkaloids and/or volatile oils extracted from 30 g of Trichosanthes kirilowii and total alkaloids, and/or volatile oils extracted from 20 g of Atractylodes lancea.

(2) The onset of severe symptoms is the same as that of typical influenza: the cough worsens sharply after 1-2 days, high fever, exhaustion, severe cough, bloody sputum, secondary shortness of breath and cyanosis. Respiratory and circulatory failure and death often occur within 1-2 weeks.

The composition of the active pharmaceutical ingredients (dry product) of this example is as follows: total alkaloids and/or volatile oils extracted from 30 g of Gastrodia elata, total alkaloids and/or volatile oils extracted from 25 g of Pinellia ternata, total alkaloids and/or volatile oils extracted from 20 g of Orange Peel, total alkaloids and/or volatile oils extracted from 30 g of Uncaria rhynchophylla, total alkaloids and/or volatile oils extracted from 30 g of Fritillaria cirrhosa, total alkaloids and/or volatile oils extracted from 30 g of Trichosanthes kirilowii, total alkaloids and/or volatile oils extracted from 15 g of Semen Lepidii, total alkaloids and/or volatile oils extracted from 30 g of Garlic, and total alkaloids and/or volatile oils extracted from 20 g of Cirsium setosum.

When the high fever subsides and respiratory and circulatory failure occurs, the composition of the active pharmaceutical ingredients (dry product) of this example is as follows: total alkaloids and/or volatile oils extracted from 30 g of Gastrodia elata, total alkaloids and/or volatile oils extracted from 30 g of Pinellia temata, total alkaloids and/or volatile oils extracted from 20 g of Orange Peel, total alkaloids and/or volatile oils extracted from 20 g of Uncaria rhynchophylla, total alkaloids and/or volatile oils extracted from 25 g of Rhizoma arisaematis, total alkaloids and/or volatile oils extracted from 20 g of Sinapis alba, total alkaloids and/or volatile oils extracted from 20 g of Radix Stemma, total alkaloids and/or volatile oils extracted from 30 g of Aconitum caunichaeli, total alkaloids and/or volatile oils extracted from 30 g of Astragalus membranaceus, total alkaloids and/or volatile oils extracted from 15 g of Angelica sinensis, total alkaloids and/or volatile oils extracted from 20 g of Ligusticum wallichii, and total alkaloids and/or volatile oils extracted from 20 g of Atractylodes lancea.

When passing through the critical period and entering the rehabilitation period, the composition of the active pharmaceutical ingredients (dry product) of this example is as follows: total alkaloids and/or volatile oils extracted from 20 g of Gastrodia elata, total alkaloids and/or volatile oils extracted from 20 g of Pinellia ternata, total alkaloids and/or volatile oils extracted from 15 g of Orange Peel, total alkaloids and/or volatile oils extracted from 15 g of Uncaria rhynchophylla, total alkaloids and/or volatile oils extracted from 15 g of Sinapis alba, total alkaloids and/or volatile oils extracted from 20 g of Astragalus membranaceus, total alkaloids and/or volatile oils extracted from 10 g of Angelica sinensis, total alkaloids and/or volatile oils extracted from 10 g of Ligusticum wallichii, and total alkaloids and/or volatile oils extracted. from 15 g of Atractylodes lancea.

### Example 2

This example is used to treat influenza caused by various coronaviruses. Specifically, it is aimed at the formulation of medicine designed for treating novel coronavirus 2019-COVID. The formulation is optimized by referring to the clinical manifestations and traditional Chinese medicine treatment in the “Diagnosis and Treatment Protocol for Novel Coronavirus Pneumonia” (Trial Version 5) published in China.

1. Medical observation period: the clinical manifestations are fatigue with gastrointestinal discomfort or fatigue with fever.

The composition of the active pharmaceutical ingredients (dry product) of this example is as follows: total alkaloids and/or volatile oils extracted from 15 g of Gastrodia elata, total alkaloids and/or volatile oils extracted from 15 g of Pinellia ternata, total alkaloids and/or volatile oils extracted from 10 g of Orange Peel, total alkaloids and/or volatile oils extracted from 15 g of Uncaria rhynchophylla.

2. Clinical treatment period: (1) the initial clinical manifestations are fever with aversion to cold or apyrexia, dry cough, dry throat, general fatigue, chest tightness, abdominal distension or vomiting, diarrhea, light red tongue, white and greasy tongue coating.

The composition of the active pharmaceutical ingredients (dry product) of this example is as follows: total alkaloids and/or volatile oils extracted from 20 g of Gastrodia elata, total alkaloids and/or volatile oils extracted from 20 g of Pinellia. ternata, total alkaloids and/or volatile oils extracted from 15 g of Orange Peel, total alkaloids and/or volatile oils extracted from 20 g of Uncaria rhynchophylla, total alkaloids and/or volatile oils extracted from 15 g of Agastache rugosa, total alkaloids and/or volatile oils extracted from 15 g of Atractylodes lancea, total alkaloids and/or volatile oils extracted from 15 g of Ginger, and total alkaloids and/or volatile oils extracted from 10 g of Notopterygium incisum.

(2) The intermediate clinical manifestations are high fever, cough with less phlegm, or yellow phlegm, abdominal distension and constipation, chest tightness, shortness of breath and panting, asthma, red tongue, yellow and greasy tongue coating.

The composition of the active pharmaceutical ingredients (dry product) of this example is as follows: total alkaloids and/or volatile oils extracted from 30 g of Gastrodia elata, total alkaloids and/or volatile oils extracted from 20 g of Pinellia ternata, total alkaloids and/or volatile oils extracted from 10 g of Orange Peel, total alkaloids and/or volatile oils extracted from 30 g of Uncaria rhynchophylla, total alkaloids and/or volatile oils extracted from 15 g of Atractylodes lancea, total alkaloids and/or volatile oils extracted from 15 g of Magnolia officinalis, total alkaloids and/or volatile oils extracted from 30 g of Fritillaria cirrhosa, total alkaloids and/or volatile oils extracted from 30 g of Trichosanthes kirilowii, total alkaloids and/or volatile oils extracted from 15 g of Semen Lepidii, and total alkaloids and/or volatile oils extracted from 15 g of Garlic.

(3) The severe clinical manifestations are dyspnea, asthma, oxygen lack with coma, irritability, sweating cold limbs, dark purple tongue, thick greasy or dry tongue coating.

The composition of the active pharmaceutical ingredients (dry product) of this example is as follows: total alkaloids and/or volatile oils extracted from 35 g of Gastrodia elata, total alkaloids and/or volatile oils extracted from 35 g of Pinellia ternata, total alkaloids and/or volatile oils extracted from 30 g of Orange Peel, total alkaloids and/or volatile oils extracted from 20 g of Uncaria rhynchophylla, total alkaloids and/or volatile oils extracted from 25 g of Rhizoma arisaematis, total alkaloids and/or volatile oils extracted from 20 g of Sinapis alba, total alkaloids and/or volatile oils extracted from 20 g of Radix Stemma, total alkaloids and/or volatile oils extracted from 20 g of Aconitum carmichaeli, total alkaloids and/or volatile oils extracted from 30 g of Astragalus membranaceus, total alkaloids and/or volatile oils extracted from 15 g of Angelica sinensis, total alkaloids and/or volatile oils extracted from 20 g of Ligusticum wallichii, total alkaloids and/or volatile oils extracted from 15 g of Achyranthes bidentata, and total alkaloids and/or volatile oils extracted from 15 g of Herba artemisiae scopariae.

(4) The clinical manifestations in the recovery period are short breath, fatigue, loss of appetite, vomiting, abdominal distension, loose stool, white and greasy tongue coating.

The composition of the active pharmaceutical ingredients (dry product) of this example is as follows: total alkaloids and/or volatile oils extracted from 15 g of Gastrodia elata, total alkaloids and/or volatile oils extracted from 15 g of Pinellia ternata, total alkaloids and/or volatile oils extracted from 15 g of Orange Peel, total alkaloids and/or volatile oils extracted from 10 g of Uncaria rhynchophylla, total alkaloids and/or volatile oils extracted from 15 g of Atractylodes lancea, total alkaloids and/or volatile oils extracted from 15 g of Magnolia officinalis, total alkaloids and/or volatile oils extracted from 30 g of Astragalus membranaceus, total alkaloids and/or volatile oils extracted from 15 g of Angelica sinensis, and total alkaloids and/or volatile oils extracted from 15 g of Radix angelicae pubescentis.

Technical effect analysis: during the treatment of pneumonia caused by Wuhan novel coronavirus in 2020, the treatment of patients by using the combination of traditional Chinese medicine and Western medicine in the initial and intermediate periods was obviously more effective compared to the treatment of patients by using only Western medicine. The Chinese medicine prescriptions published in the fifth edition are as follows:

1. Medical observation period: Huoxiang Zhengqi Capsules (water, oral liquid) or Jinhua Qingre Granules are used.

2. Clinical treatment period: (1) the initial period prescription: Atractylodes lancea 15 g, Dried Orange Peel 10 g, Magnolia officinalis 10 g, Agastache rugosa 10 g, Lanxangia tsao-ko 6 g, raw Chinese ephedra 6 g, Notopterygium incisum 10 g, Ginger 10 g, Areca catechu 10 g.

(2) The medium period prescription: Almond 10 g, Gypsum 30 g, Trichosanthes kiriiowii 30 g, raw Rheum officinale 6 g (decocted later), raw Chinese ephedra 6 g, Semen Lepidii 10 g, Peach Kernel 10 g, Lanxangia tsao-ko 6 g, Areca catechu 10 g and Atractylodes lancea 10 g.

(3) The severe period prescription: Ginseng 15 g, black prepared lateral root of aconite (Aconitum camiichaeli) 10 g, Cornus officinalis 15 g.

3. The prescription in the recovery period: Rhizoma pineliae praeparata 9 g, Dried Orange Peel 10 g, Codonopsis pilosula 10 g, Astragalus membranaceus 30 g, Poria cocos 15 g, Agastache rugosa 10 g, Amomum villosum (decocted later) 6 g,

According to the analysis of the four compositions, the drugs mainly used are dampness resolving drugs, antiasthmatic drugs, phlegm resolving drugs, etc., so they are effective for mild- or medium-period patients, but not for severe-period patients. Their main disadvantage is the lack of drugs that can pass through the blood-brain barrier. The medicine provided by the invention is added with drugs such as Gastrodia data, Uncaria rhynchophylla and Rhizoma arisaematis that can pass through blood-brain barrier, so the technical effect will be significantly improved compared to that of the above-mentioned compositions. Because the symptoms of “oxygen lack with coma” are obvious symptoms of brain infections, and viruses or fungi often easily invade the brain tissues, the development of the disease can't be controlled in the middle and late periods without the treatment of the brain infections. Moreover, the medicine provided by the invention is manufactured by extracting alkaloids and/or volatile oils from various plants, and the chemical ingredients are clear. Compared to traditional Chinese medicine decoctions, the technical effect of the medicine of the invention is obvious.

### Example 3

The example is a medicine for treating infectious diseases caused by Cryptococcus or AIDS.

1. The first clinical symptoms are the symptoms of upper respiratory tract infections such as chills, fever, headache, dizziness, sore throat, conjunctivitis, nasal congestion, nausea, vomiting, general discomfort and other symptoms.

The composition of the active pharmaceutical ingredients (dry product) of this example is as follows: total alkaloids and/or volatile oils extracted from 30 g of Gastrodia elata, total alkaloids and/or volatile oils extracted from 30 g of Pinellia ternata, total alkaloids and/or volatile oils extracted from 25 g of Orange Peel, total alkaloids and/or volatile oils extracted from 30 g of Uncaria rhynchophylla, total alkaloids and/or volatile oils extracted from 15 g of Ginger, total alkaloids and/or volatile oils extracted from 10 g of Notopterygium incisum, total alkaloids and/or volatile oils extracted from 15 g of Ligusticum wallichii, total alkaloids and/or volatile oils extracted from 20 g of Mentha haplocalyx, total alkaloids and/or volatile oils extracted from 20 g of Chaste Tree Fruits, total alkaloids and/or volatile oils extracted from 15 g of Ligusticum wallichii, and total alkaloids and/or volatile oils extracted from 20 g of Atractylodes lancea.

2. When the patient's condition develops to severe eye symptoms or mental symptoms or central nervous system injury or general symptoms, the composition of the active pharmaceutical ingredients (dry product) of this example is as follows: total alkaloids and/or volatile oils extracted from 50 g of Gastrodia elata, total alkaloids and/or volatile oils extracted from 30 g of Pinellia ternata, total alkaloids and/or volatile oils extracted from 25 g of. Orange Peel, total alkaloids and/or volatile oils extracted from 50 g of Uncaria rhynchophylla, total alkaloids and/or volatile oils extracted from 30 g of Rhizoma arisaematis, total alkaloids and/or volatile oils extracted from 20 g of Aconitum carmichaeli, total alkaloids and/or volatile oils extracted from 25 g of Ligusticum wallichii, total alkaloids and/or volatile oils extracted from 25 g of Ginger, total alkaloids and/or volatile oils extracted from 15 g of Notopterygium incisum, total alkaloids and/or volatile oils extracted from 20 g of Radix angelicae pubescentis, total alkaloids and/or volatile oils extracted from 20 g of Achyranthes bidentata, and total alkaloids and/or volatile oils extracted from 20 g of Atractylodes lancea.

### Example 4

The example is a medicine for treating infectious diseases caused by Mucor or Entomophthora or Ebola, virus. The clinical symptoms are headache, nasal discharge stained with blood, eye congestion, tumor of one side of the face, nerve palsy, chest tightness, chest pain, cough and bloody sputum. The onset of the disease is acute, it can progress to systemic hemorrhage soon, and can cause death within 2 weeks.

The composition of the active pharmaceutical ingredients (dry product) of this example is as follows: total alkaloids and/or volatile oils extracted from 50 g of Gastrodia elata, total alkaloids and/or volatile oils extracted from 50 g of Pinellia ternata, total alkaloids and/or volatile oils extracted from 50 g of Orange Peel, total alkaloids and/or volatile oils extracted from 50 g of Uncaria rhynchophylla, total alkaloids and/or volatile oils extracted from 50 g of Rhizoma arisaematis, total alkaloids and/or volatile oils extracted from 50 g of Aconitum carmichaeli, total alkaloids and/or volatile oils extracted from 50 grams of Astragalus membranaceus, total alkaloids and/or volatile oils extracted from 50 g of Cirsium setosum, and total alkaloids and/or volatile oils extracted from 50 g of Cirsium japonicum.

Of course, the examples above are only for the purpose of clearly illustrating the examples given, rather than limiting the implementation mode. Based upon the above description, other different changes or modifications can be also made by those skilled in the art. It is unnecessary and impossible to enumerate all the examples here, and the obvious changes or variations thus extended are still within the protection scope of the present invention.

### Preparation Methods

The preparation method 1 is as follows: according to the weight parts of the selected raw materials in the above examples, alkaloids and volatile oils are extracted from each of the raw materials using modern conventional extraction methods; the alkaloids are mixed, and added with conventional excipients; pharmaceutically acceptable dosage forms such as tablets or powder injections are prepared according to conventional processes for preparing medicines.

Then, the volatile oils are mixed, and added with conventional excipients; pharmaceutically acceptable dosage forms such as soft capsules or granules and topical liniments are prepared according to conventional processes for preparing medicines.

The volatile oils are mixed in a proportion and then mixed with the dry alkaloid substances and an appropriate amount of excipients after inclusion with beta-cyclodextrin; pharmaceutically acceptable dosage forms such as tablets, granules are prepared using conventional methods for preparing medicines.

The preparation method 2 is as follows: according to the weight parts of the selected raw materials in the above examples, alkaloids contained in Gastrodia elata, Pinellia ternata, Orange Peel and Uncaria rhynchophylla are extracted using conventional methods and added with conventional excipients, and pharmaceutically acceptable dosage forms such as tablets or powder injections are prepared according to conventional processes for preparing medicines; then, alkaloids extracted from additional raw materials are mixed, and added with conventional excipients; pharmaceutically acceptable dosage forms such as tablets or powder injections are prepared according to conventional processes for preparing medicines, and an A+B combination is obtained.

Then, volatile oils extracted from Gastrodia elata, Pinellia temata, Orange Peel and Uncaria rhynchophylla are mixed, and added with conventional excipients; pharmaceutically acceptable dosage forms such as soft capsules or granules are prepared according to conventional processes for preparing medicines; then, volatile oils extracted from additional raw materials are mixed, and added with conventional excipients; pharmaceutically acceptable dosage forms such as soft capsules or granules are prepared according to conventional processes for preparing medicines, and an A+B combination is obtained.

Alternatively, volatile oils extracted from Gastrodia elata, Pinellia ternata, Orange Peel and Uncaria rhynchophylla are mixed in a proportion and then mixed with the dry alkaloid substances and an appropriate amount of excipients after inclusion with beta-cyclodextrin; pharmaceutically acceptable dosage forms such as tablets, granules are prepared using conventional methods for preparing medicines; then, volatile oils extracted from additional raw materials are mixed in a proportion and then mixed with the dry alkaloid substances and an appropriate amount of excipients after inclusion with beta-cyclodextrin; pharmaceutically acceptable dosage forms such as tablets, granules are prepared using conventional methods for preparing medicines, and an A+B combination is obtained.

The preparation method 3 is as follows: total alkaloids and/or total volatile oils are extracted from corresponding fresh raw materials and then grouped to prepare medicine, wherein a standard dose of 15 g of the dry product is used for each of the raw materials. The alkaloids in each of the groups are mixed, and added with conventional excipients; pharmaceutically acceptable dosage forms such as tablets or powder injections are prepared according to conventional processes for preparing medicines.

Then, the volatile oils in each of the groups are mixed, and added with conventional excipients; pharmaceutically acceptable dosage forms such as soft capsules or granules and topical liniments are prepared according to conventional processes for preparing medicines.

Furthermore, the volatile oils in each of the groups are mixed in a proportion and then mixed with the dry alkaloid substances and an appropriate amount of excipients after inclusion with beta-cyclodextrin; pharmaceutically acceptable dosage forms such as tablets, granules are prepared using conventional methods for preparing medicines. The specific grouping is as follows:

The first group: total alkaloids and/or volatile oils extracted from 15 g of Gastrodia elata, total alkaloids and/or volatile oils extracted from 15 g of Pinellia ternata, total alkaloids and/or volatile oils extracted from 15 g of Orange Peel, total alkaloids and/or volatile oils extracted from 15 g of Uncaria rhynchophylla.

The second group: total alkaloids and/or volatile oils extracted from 15 g of Ginger, total alkaloids and/or volatile oils extracted from 15 g of Notopterygium incisum, total alkaloids and/or volatile oils extracted from 15 g of Saposhnikovia divaricata, total alkaloids and/or volatile oils extracted from 15 g of Ligusticum levisticum, total alkaloids and/or volatile oils extracted from 15 g of Flos Magnoliae.

The third group: total alkaloids and/or volatile oils extracted from 15 g of Mentha haplocalyx, total alkaloids and/or volatile oils extracted from 15 g of Mulberry Leaves, total alkaloids and/or volatile oils extracted from 15 g of Chaste Tree Fruits.

The fourth group: total alkaloids and/or volatile oils extracted from 15 g of Agastache rugosa, total alkaloids and/or volatile oils extracted from 15 g of Atractylodes lancea, total alkaloids and/or volatile oils extracted from 15 g of Magnolia officinalis, total alkaloids and/or volatile oils extracted from 15 g of Garlic.

The fifth group: total alkaloids and/or volatile oils extracted from 15 g of Rhizoma. arisaematis, total alkaloids and/or volatile oils extracted from 15 g of Sinapis alba, total alkaloids and/or volatile oils extracted from 15 g of Radix Stemona.

The sixth group: total alkaloids and/or volatile oils extracted from 15 g of Fritillaria cirrhosa, total alkaloids and/or volatile oils extracted from 15 g of Trichosanthes kirilowii, total alkaloids and/or volatile oils extracted from 15 g of Semen Lepidii.

The seventh group: total alkaloids and/or volatile oils extracted from 15 g of Radix angelicae pubescentis, total alkaloids and/or volatile oils extracted from 15 g of Achyranthes bidentata, total alkaloids and/or volatile oils extracted from 15 g of Aconitum carmichaeli, total alkaloids and/or volatile oils extracted from 15 g of Herba artemisiae scopariae.

The eighth group: total alkaloids and/or volatile oils extracted from 15 g of Astragalus membranaceus, total alkaloids and/or volatile oils extracted from 15 g of Angelica sinensis, total alkaloids and/or volatile oils extracted from 15 g of Ligusticum wallichii.

The ninth group: total alkaloids and/or volatile oils extracted from 15 g of Cirsium setosum, total alkaloids and/or volatile oils extracted from 15 g of Cirsium japonicum.

### Technical Effect

The traditional prescriptions such as the “Banxia Baizhu Tianma Decoction”, “Er Chen Decoction” and “Yu Hu Pill”, as well as other plant medicines have been clinically proved to be safe and effective. The medicine provided by the invention is based on raw materials of traditional medicine prescriptions, wherein total alkaloids and/or volatile oils contained in plants such as Gastrodia elata, Pinellia ternata, Orange Peel, Unearia rhynchophylla are extracted to prepare medicines or health products for treating influenza or infectious diseases. Since the chemical ingredients of a medicine or health product provided by the invention are definite, their pharmacological action is clear, and the effectiveness thereof is obvious and predictable when comparing traditional methods for preparing drugs with the methods for preparing modern Chinese patent medicines.

During the treatment of pneumonia caused by novel coronavirus in 2020, it was proved that traditional decoctions can have a better therapeutic effect on patients with mild symptoms. It was reported at a news conference in China that chloroquine phosphate had a certain diagnosis and treatment effect on the new coronavirus. Chloroquine phosphate is phosphate of chloroquine, and chloroquine is derived from the structural optimization of quinine, wherein quinine is an alkaloid extracted from a plant such as Cinchona. It is proved that alkaloids have an effect against novel coronavirus.

Because the applicant has no conditions to extract alkaloids and/or volatile oils from the raw materials for clinical trials, but those skilled in the art are able to implement the technical solution of the invention based on the contents recorded in this description and the common technical knowledge in the art to solve the technical problems and provide the expected technical effect.

The inventive step of the medicine provided by the invention is as follows: the active pharmaceutical ingredients are total alkaloids and/or volatile oils extracted from Gastrodia elata, total alkaloids and/or volatile oils extracted from Pinellia ternata, total alkaloids and/or volatile oils extracted from Orange Peel, total alkaloids and/or volatile oils extracted from Uncaria rhynchophylla, etc. It is clear that the chemical ingredients of each of the raw materials are alkaloids and volatile oils. Compared with the existing pharmaceutical chemicals which are medicines made from alkaloids extracted from a single plant, the medicine provided by the invention can reduce the content of a single alkaloid in a medicine, reduce the toxic reaction caused by the excessive dosage of the single alkaloid, and improve the drug tolerability. Moreover, the medicines provided by the invention can have a more effective targeting action on viral or fungal infections in different parts due to the synergistic effect of multiple alkaloids, and has more drug resistance. Because the inventor thinks that the immunity of human body comes from the resistance to Candida albicans in the body, and the medicine provided by the invention acts against Candida albicans, so the medicine provided by the invention also has immunosuppressive action.

Furthermore, compared with traditional Chinese medicines prepared using modern methods for preparing drugs, the medicines provided by the present invention show significant progress. Because not all Chinese medicines are necessarily obtained from herbs, but also from animals and minerals, the most important feature of modern Chinese medicines is that it is possible to know only the main ingredients, rather than to know all the ingredients, and the pharmacological mechanism of the existing Chinese patent medicines is not clear; and the chemical ingredients of the medicine provided by the invention are only alkaloids and volatile oils, so the chemical ingredients are definite. The pharmacological mechanism of the medicine provided by the invention is clear, wherein its pharmacological mechanism is that the total alkaloids of each of the raw materials have antifungal and antiviral effects, as well as the total volatile oils of each of the raw materials have antifungal and antiviral effects. Moreover, these alkaloids and volatile oils have their own characteristics, and can specifically target fungal or viral infections in different blood vessels, tissues and cells.

Therefore, the medicine provided by the invention has clear pharmacological mechanism and definite chemical ingredients. Compared with the medicines of modern Western medicine that are made of a single alkaloid, it is characterized by obvious curative effect, as well as by anti-drug resistance and weaker toxic side reactions. Compared with traditional Chinese medicines and modern Chinese patent medicines, the invention shows significant progress. The technical solution provided by the invention uses the advantages of drug combination therapy of traditional Chinese medicine, as well as the advantages of modern pharmaceutical chemicals, overcomes the shortcoming that the effective chemical ingredients and pharmacological mechanism of traditional Chinese medicine prescriptions are not clear, and also overcomes the disadvantages of modern Western medicine that the chemical ingredient is single, thereby easily causing side effects, toxic reactions and drug resistance.

1. A medicine for treating influenza or infectious diseases caused by viruses or fungi, characterized in that the medicine is used for treating influenza or infectious diseases caused by influenza viruses, coronaviruses, Ebola virus, AIDS, Aspergillus, Cryptococcus, Mucor, Entomophthora, Histoplasma capsulatum, wherein the active pharmaceutical ingredients of the medicine include total alkaloids and/or total volatile oils extracted from Gastrodia elata, total alkaloids and/or total volatile oils extracted from Pinellia ternata, total alkaloids and/or total volatile oils extracted from Orange Peel, and total alkaloids and/or total volatile oils extracted from Uncaria rhynchophylla, wherein the methods for preparing the medicine include: taking the total alkaloids of Gastrodia elata, the total alkaloids of Pinellia ternata, the total alkaloids of Orange Peel and the total alkaloids of Uncaria rhynchophylla and mixing them in a certain proportion; adding various conventional excipients required to prepare different dosage forms thereto; and preparing pharmaceutically acceptable dosage forms; taking the total volatile oils of Gastrodia elata, the total volatile oils of Pinellia ternata, the total volatile oils of Orange Peel and the total volatile oils of Uncaria rhynchophylla and mixing them in a certain proportion; adding various conventional excipients required to prepare different dosage forms thereto; and preparing pharmaceutically acceptable dosage forms; taking the total alkaloids and total volatile oils of Gastrodia elata, the total alkaloids and total volatile oils of Pinellia ternata, the total alkaloids and total volatile oils of Orange Peel, the total alkaloids and total volatile oils of Uncaria rhynchophylla; mixing the volatile oils in a proportion, then mixing them with the dry alkaloid substances and an appropriate amount of excipients after inclusion with beta-cyclodextrin, and preparing pharmaceutically acceptable dosage forms using conventional methods for preparing medicines.

2. The medicine according to claim 1, characterized in that when the clinical symptoms appear to be upper respiratory tract infection symptoms, the active pharmaceutical ingredients of the medicine can be selectively added with: total alkaloids and/or total volatile oils extracted from Ginger, total alkaloids and/or total volatile oils extracted from Notopterygium incisum, total alkaloids and/or total volatile oils extracted from Saposhnikovia divaricata, total alkaloids and/or total volatile oils extracted from Ligusticum levisticum, total alkaloids and/or total volatile oils extracted from Flos Magnoliae, total alkaloids and/or total volatile oils extracted from Mentha haplocalyx, total alkaloids and/or total volatile oils extracted from Mulberry Leaves, total alkaloids and/or total volatile oils extracted from Chaste Tree Fruits.

3. The medicine according to claim 1, characterized in that when the clinical symptoms appear to be digestive system symptoms, the active pharmaceutical ingredients of the medicine can be selectively added with: total alkaloids and/or total volatile oils extracted from Agastache rugosa, total alkaloids and/or total volatile oils extracted from Atractylodes lancea, total alkaloids and/or total volatile oils extracted from Magnolia officinalis.

4. The medicine according to claim 1, characterized in that when the clinical symptoms appear to be pneumonia symptoms, the active pharmaceutical ingredients of the medicine can be selectively added with: total alkaloids and/or total volatile oils extracted from Rhizoma arisaematis, total alkaloids and/or total volatile oils extracted from Sinapis alba, total alkaloids and/or total volatile oils extracted from Fritillaria cirrhosa, total alkaloids and/or total volatile oils extracted from Trichosanthes kirilowii, total alkaloids and/or total volatile oils extracted from Radix Stemona, and total alkaloids and/or total volatile oils extracted from Semen Lepidii.

5. The medicine according to claim 1, characterized in that when the clinical symptoms appear to be general symptoms, the active pharmaceutical ingredients of the medicine can be selectively added with: total alkaloids and/or total volatile oils extracted from Radix angelicae pubescentis, total alkaloids and/or total volatile oils extracted from Ligusticum wallichii, total alkaloids and/or total volatile oils extracted from Achyranthes bidentata, total alkaloids and/or total volatile oils extracted from Astragalus membranaceus, total alkaloids and/or total volatile oils extracted from Angelica sinensis, total alkaloids and/or total volatile oils extracted from Aconitum carmichaeli, total alkaloids and/or total volatile oils extracted from Herba artemisiae scopariae, total alkaloids and/or total volatile oils extracted from Garlic, total alkaloids and/or total volatile oils extracted from Cirsium setosum, and total alkaloids and/or total volatile oils extracted from Cirsium japonicum.

6. The medicine according to claims 1, characterized in that the methods for preparing the medicine include: taking Gastrodia elata, Pinellia ternata, Orange Peel and Uncaria rhynchophylla as the basic raw materials, selectively adding them with raw materials according to the clinical symptoms, extracting the respective total alkaloids and total volatile oils from the raw materials according to the weight parts of the selected raw materials by using modern conventional extraction methods, mixing the total alkaloids and adding conventional excipients thereto, and preparing pharmaceutically acceptable dosage forms such as tablets or powder injections according to conventional processes for preparing medicines; mixing the respective total volatile oils and adding conventional excipients thereto, and preparing pharmaceutically acceptable dosage forms according to conventional processes for preparing medicines; mixing the volatile oils in a proportion and then mixing them with the dry alkaloid substances and an appropriate amount of excipients after inclusion with beta-cyclodextrin, and preparing pharmaceutically acceptable dosage forms such as tablets, granules using conventional methods for preparing medicines.

7. The medicine according to claim 1, characterized in that the methods for preparing the medicine include: according to the weight parts of the selected raw materials, mixing the total alkaloids and/or total volatile oil extracted from Gastrodia elata, Pinellia ternata, Orange Peel and Uncaria rhynchophylla by using modern conventional extraction methods; mixing the total alkaloids and/or total volatile oils extracted from the raw materials added according to the clinical symptoms, and adding conventional excipients thereto; preparing pharmaceutically acceptable dosage forms according to conventional processes for preparing medicines; and obtaining an A+B combination.

8. The medicine according to claim 1, characterized in that the methods for preparing the medicine include: extracting total alkaloids and/or total volatile oils from the corresponding fresh raw materials and grouping them to prepare medicine, wherein a standard dose of 15 g of the dry product is used for each of the raw materials; the specific groups are as follows: (1) Gastrodia elata, Pinellia ternata, Orange Peel and Uncaria rhynchophylla; (2) Ginger, Notopterygium incisum, Saposhnikovia divaricata, Ligusticum levisticum and Flos Magnoliae; (3) Mentha haplocalyx, Mulberry leaves and Chaste Tree Fruits; (4) Agastache rugosa, Atractylodes lancea, Magnolia officinalis, Garlic and/or Herba artemisiae scopariae; (5) Rhizoma arisaematis, Sinapis alba and Radix Stemona; (6) Fritillaria cirrhosa, Trichosanthes kirilowii and Semen Lepidii; (7) Radix angelicae pubescentis, Achyranthes bidentata, Aconitum carmichaeli; (8) Astragalus membranaceus, Angelica sinensis and Ligusticum wallichii; (9) Cirsium setosum and Cirsium japonicum; wherein the extracted total alkaloids in small groups are used to prepare pharmaceutically acceptable dosage forms; the extracted total volatile oils in small groups are used to prepare pharmaceutically acceptable dosage forms; and the extracted total alkaloids and total volatile oils in small groups are used to prepare pharmaceutically acceptable dosage forms.

9. The medicine according to claim 1, characterized in that the scope of application of the medicine to treat influenza or infectious diseases caused by influenza viruses or HN avian influenza virus, coronaviruses or coronavirus 2019-COVID or MERS virus or SARS coronavirus, Ebola virus, AIDS, Aspergillus, Cryptococcus, Mucor, Entomophthora and Histoplasma capsulatum includes applications in medical drugs and health products.
